(19) United States
(12) Patent Application Publication
(10) Pub. No.: US 2008/0228231 A1
(43) Pub. Date: Sep. 18, 2008

(54) Acoustic Back-Scattering Sensing Screw for Preventing Spine Surgery Complications

(75) Inventors: David T. Raphael, Valley Village, CA (US); K. Kirk Shung, Monterey Park, CA (US)

Correspondence Address: McDERMOTT WILL & EMERY LLP;Suite 3800, 2049 Century Park East, Los Angeles, CA, 90067, US

(73) Assignee: University of Southern California

(21) Appl. No.: 12/015,910

(22) Filed: Jan. 17, 2008

Related U.S. Application Data
(60) Provisional application No. 60/885,822, filed on Jan. 19, 2007.
(60) Provisional application No. 60/984,630, filed on Nov. 1, 2007.

(51) Int. Cl. A61B 17/04; A61N 1/00; G01H 11/06

(52) U.S. Cl. 606/301; 607/60; 367/181

(57) ABSTRACT

A medical device having an implantable instrument configured to be implanted in bone tissue, the implantable instrument having an internal chamber configured to house at least one transducer; a transducer located within the internal chamber of the implantable instrument, the transducer configured to transmit a signal and to receive one or more reflections of that signal; and a signal processing system configured to process the signals transmitted and received by the transducer so as to provide information indicative of the acoustic impedance of matter in the vicinity of the implantable instrument after it is implanted into the bone tissue based on the signals transmitted and received by the transducer.

### CROSS-REFERENCE TO RELATED APPLICATIONS

This application claims priority from provisional patent 60/885,822 (“ACOUSTIC BACK-SCATTERING SENSING SCREW FOR PREVENTING SPINE SURGERY COMPLICATIONS” Attorney Docket Number 028080-257) filed Jan. 19, 2007, and 60/984,630 (“ACOUSTIC BACK-SCATTERING SENSING SCREW FOR PREVENTING SPINE SURGERY COMPLICATIONS” Attorney Docket Number 028080-0305), filed Nov. 1, 2007, the entire disclosure of both provisional applications is incorporated by reference.

### BACKGROUND

1. Field

This application relates to the placement and positioning of implanted instruments in bone tissue to prevent nerve injury.

2. Description of Related Art

Transpedicular screw fixation is a popular spine stabilization procedure. For example transpedicular screw fixation is often used in treating degenerative spine patients undergoing lumbosacral fusion. In such procedures pedicle screws may be implanted into a vertebral pedicle in order to affix rods and plates to the spine. Pedicle screws may also be used to immobilize part of the spine to assist in bone repair and fusion by holding bony structures together.

The transpedicular screw fixation approach has a higher overall fusion rate (91%) compared to autologous bone grafting (51%), and has respectively a lower overall complication rate (13.4% versus 40.6%).

However current transpedicular screw fixation techniques may have drawbacks. In a survey analysis of 617 surgical cases in which pedicle screw implants were used with three different spinal implant systems (Esses S et al., Spine 1993; 18(5):2231-8), the most common problem was unrecognized screw misplacement (5.2%), including perforation of the distal vertebral cortex. Fracturing of the pedicle during screw insertion and cerebrospinal fluid leak occurred in 4.2% of cases. Transient neuropraxia occurred in 2.4% of cases, and permanent nerve injury occurred in 2.3% of cases.

Moreover, deformation and fracturing of the pedicle can occur when too thick a screw is inserted into the pedicle. (Defino et al., European Spine Jour 2001; 10: 325-333). Another complication results when the screw is advanced too far through the vertebral body, so that a penetration of the distal cortex occurs, (cortex perforation). Malpositioned pedicle screws can protrude through the cortex, and can cause injury to the spinal cord, thecal sac, and to spinal nerve roots.

As such, it is evident that pedicle screw placement is associated with significant intraoperative and postoperative complications.

To this end, different approaches have been employed to reduce pedicle screw placement complication rates. Most current transpedicle spinal fixation techniques rely on the identification of predefined targets with the help of anatomic landmarks and with the intraoperative use of ionizing fluoroscopic image intensifiers. For example, computerized axial tomography (CAT) scans may be useful in detecting malpositioned screws postfacto, but do not aide in real-time guiding and positioning of the screw nor in detecting an immediate break of the pedicle cortex. Moreover, postoperative CAT scans may overestimate the number of misplaced screws (Rao G, et al., J Neurosurgery 2002; 97(2 suppl):223-6). Other techniques that have also been considered in helping to reduce pedicle screw placement complications rates are fluoroscopy-based frameless stereotactic systems, CAT-scan derived volumetric multiple exposure transmission holography, electromagnetic field based image guided spine surgery, and intraosseous endoscopy. However these methods have associate drawbacks.

Due to existing complications associated with pedicle screw placement and the absence of accurate real-time pedicle screw guidance and positioning techniques, which have significant drawbacks, it is evident that a technique yet needs to be developed that is radiation-free, dynamically interactive, and capable of an extremely low complication rate.

### SUMMARY

A medical device having an implantable instrument may be configured to be implanted in bone tissue, the implantable instrument may have an internal chamber configured to house at least one transducer, so that the transducer may transmit a signal and receive one or more reflections of that signal indicative of the acoustic impedance of matter in the vicinity of the implantable instrument after it is implanted into the bone tissue. The medical device may also include a signal processing system which may process the electrical signal from the transducer and may provide real-time dynamic information indicative of the acoustic impedance of matter in the vicinity of the implantable instrument after it is implanted into the bone tissue based on the electrical signal from the transducer.

Specifically, for surgery involving the penetration of bone, such a medical device may allow dynamic measurement of the elastic properties of the tissue in direct local contact with the tip of the implantable instrument, whether it be solid (cortical) or spongy (cancellous) bone or soft tissue. Such a medical device may significantly enhance the safety of spine surgery through its near total elimination of pedicle screw perforations.

The medical device may include more than one transducer and one of the transducers may be configured to transmit a signal and another transducer may receive a reflection or reflections of the signal from the signal processing system. One of the transducers may include piezoelectric material so that the transducer may transmit an acoustic signal to the signal processing system.

The implantable instrument may include a lens on the distal tip, and the implantable instrument may be made of a titanium-based material that satisfies FDA strength and nontoxicity requirements. A piezoelectric layer (such as lithium niobate) may be incorporated into the implantable instrument as the active component of a single element transducer, which may transmit and receive acoustic two-way signals. Also, one or two matching layers and a light backing material may be used to increase transmission sensitivity.

A protective layer may be placed on top of the lens. The lens may be used to tightly focus the ultrasound beam at a distance, for example, a few millimeters away from the transducer. The lens may preferably be made of a tough material with an acoustic impedance similar to that of the bone, for example aluminum, that can withstand the force exerted by bone. The optimal center frequency is preferably in a range from 1 to 7.5 MHz. The transducing system may be held in place by an epoxy and may be removable upon implantation of the instrument. The transducer may be removed and replaced with a solid material and the transducer may be re-used in another implantable instrument. This design may allow the generation of ultrasound at a high sensitivity while maintaining a broad bandwidth.

The implantable instrument may have a plurality of apertures, through which transducer components may be advanced when the transducer is housed in the implantable instrument, and the apertures may also be configured to allow the placement of solid non-transducing components when the transducer is not present so that the implantable instrument may be advanced through the bone tissue and the signal generated by the transducer may not include strain artifacts.

The signal processing system may be configured to indicate, via a visual display or an audible alarm, that the position of the implantable instrument is approaching a desired or an undesired depth.

These, as well as other components, steps, features, objects, benefits, and advantages, will now become clear from a review of the following detailed description of illustrative embodiments, the accompanying drawings, and the claims.

### BRIEF DESCRIPTION OF THE DRAWINGS

FIGS. 1(a)-(d) illustrate a method of spine stabilization with transpedicular screws using the Protext Stabilization System (Globus Medical, Phoenixville, Pa.).

FIG. 2 illustrates the transducing medical device insertion system.

FIG. 3 illustrates how a time-gated pulse echo backscatter-sensing system operates in bone.

FIG. 4 illustrates the compact bone defined by the width of the bold-faced arrow, and bordered by regions of cancellous bone.

FIG. 5 illustrates a focusable beam (in the azimuthal and elevation directions) that provides a simultaneous mix of both forward and side imaging with improved contrast and better control of slice thickness.

FIG. 6 illustrates a water bath setup for scattering measurements and for velocity and attenuation measurements.

FIG. 7 illustrates A-mode amplitude increases, by serving as an indication of close proximity to cortex, to the point of signal saturation.

FIGS. 8(a)-(j) illustrate insertion of the implantable instrument in order to separate the axial and rotation movement of the implantable instrument from the acoustic measurement process.

### DETAILED DESCRIPTION OF ILLUSTRATIVE EMBODIMENTS

The drawings disclose illustrative embodiments. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

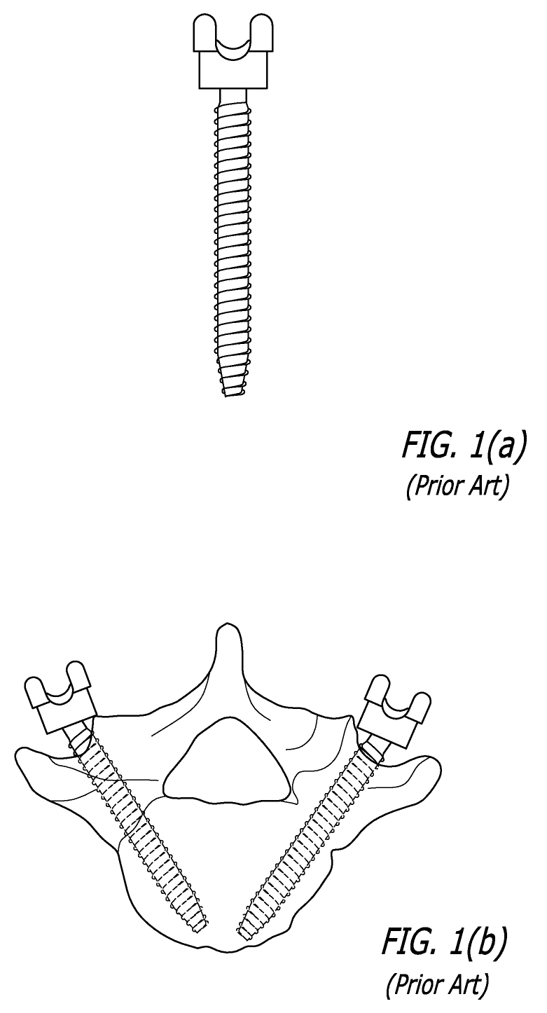

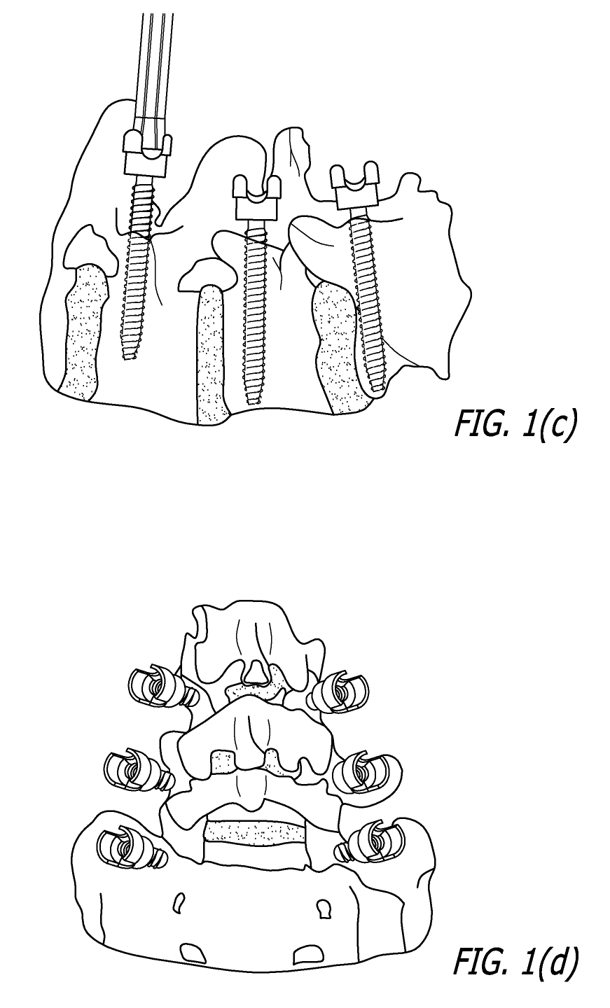

FIGS. 1(a)-(d) illustrate a method of spine stabilization with transpedicular screws using the Protext Stabilization System (Globus Medical, Phoenixville, Pa.). The pedicle screws are typically of 5.0-9.0 mm in diameter and have lengths of 30 mm to 65 mm. As shown, the screws inserted into the vertebral pedicles and may come in contact with the spinal cord.

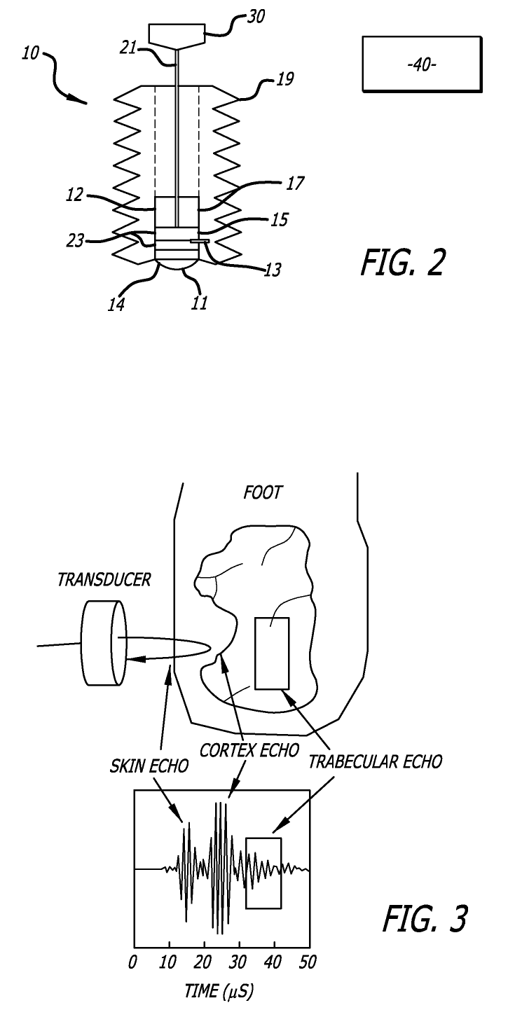

FIG. 2 illustrates a transducing implantable medical device 1. The transducing implantable medical device 1, may comprise an implantable instrument 10, which may be made of a titanium-based material that satisfies FDA strength and nontoxicity requirements.

The transducing medical device 1 may further comprise a signal processing system 40 which may be configured to process electrical signals from a transducer 12 via positive electrical lead 21 and advancement device 30. The signal may provide information indicative of the acoustic impedance of matter in the vicinity of the implantable instrument after it is implanted into the bone tissue based on the electrical signal from the transducer 12.

The implantable instrument 10 may be in the form of a screw and the advancement device 30 may be a screwdriver, and the electrical lead 21 from the transducer 12 may be directed through the internal chamber of the implantable instrument 10 and may be directed externally through an insulated cord to the proximal end of the implantable instrument 10, which in turn may be interfaced with the distal tip of the advancement device 30. The advancement device 30 may function as a means for transmitting signals through an A/D converter for signal processing.

The advancement device 30 may be configured to engage with the implantable instrument 10 to advance the implantable instrument 10 through the bone tissue. The implantable instrument 10 may include a threaded external wall 19. Examples of the type of the implantable instrument 10, may include a screw, awl, pedicle probe, pedicle ‘feel’ probe, or pedicle screw tap.

The implantable instrument 10 may further include an internal chamber configured to house at least one transducer 12. A piezoelectric layer 15 (such as lithium niobate) may be incorporated into the implantable instrument 10 as the active component of the transducer 12. The transducer 12 may transmit and receive acoustic two-way signals or may be configured to include a transducer to transmit a signal and another transducer to receive a reflection or reflections of the signal from a signal processing system 40. One or two matching layers 23 and a light backing material 17 may be used to increase transmission sensitivity of the transducer 12.

The implantable instrument 10 may include a protective layer 14 on top of the lens 11 of the transducer 12. The lens 11 may be used to tightly focus an ultrasound beam generated by the transducer 12 at a distance, for example, a few millimeters away from the transducer. The lens 11 is preferably made of a tough material with an acoustic impedance similar to that of the bone, for example aluminum, that can withstand the force exerted by bone. The optimal center frequency is preferably to range from 1 to 7.5 MHz. The transducer 12 may be held in place by an epoxy. This design may allow the generation of ultrasound at a high sensitivity while maintaining a broad bandwidth.

The signal processing system 40 may include a visual display configured to display an image of the bone tissue in the vicinity of the implantable instrument 10, for example a contour surface map of the bone tissue in the vicinity of the implantable instrument 10.

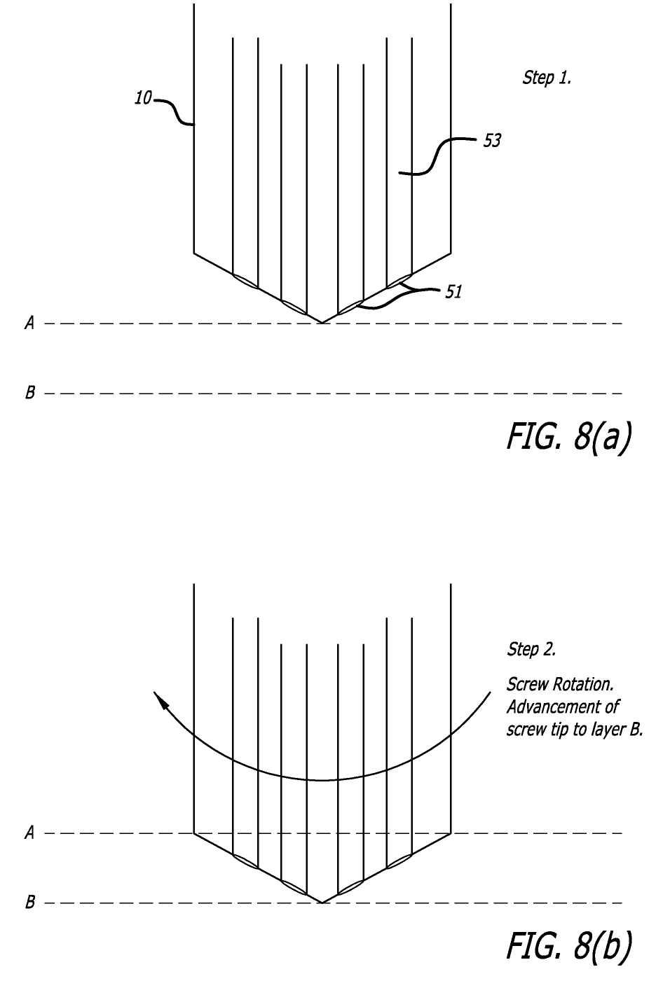

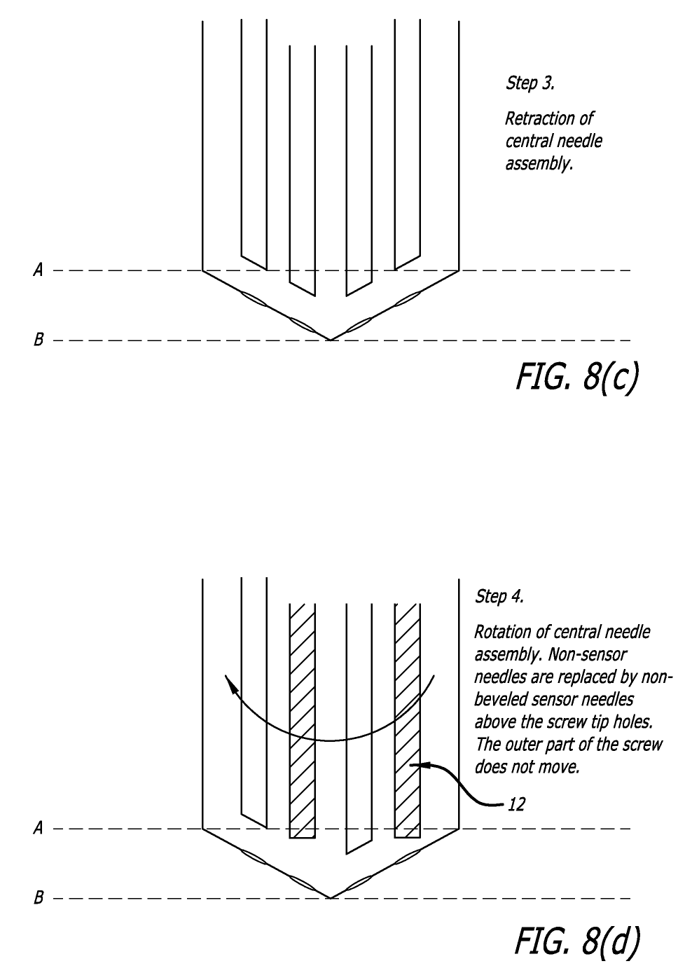

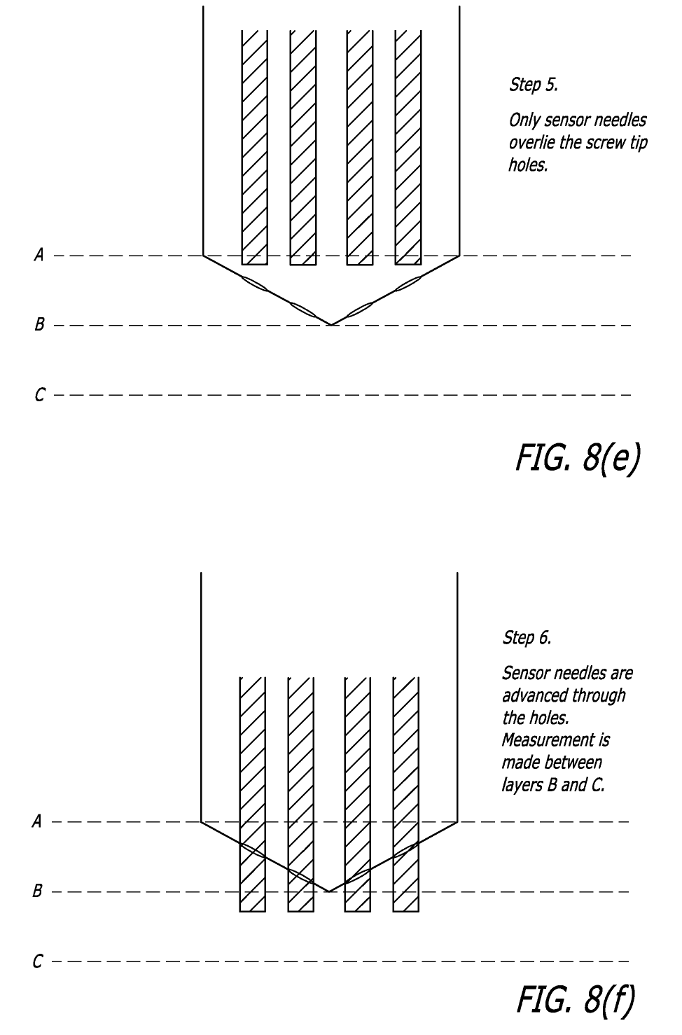

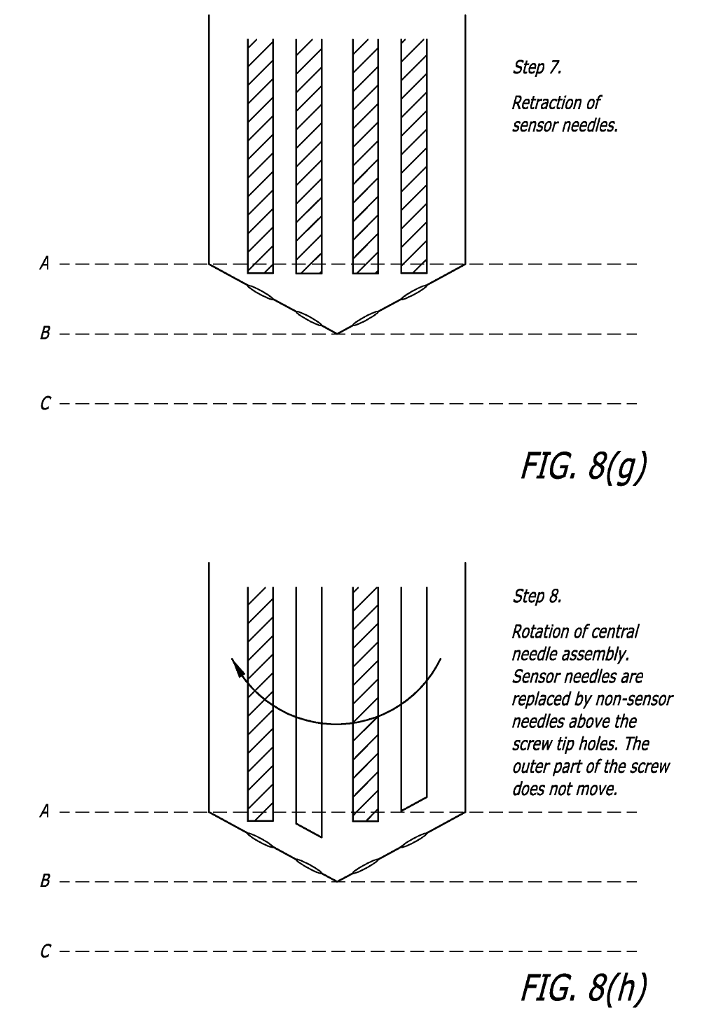

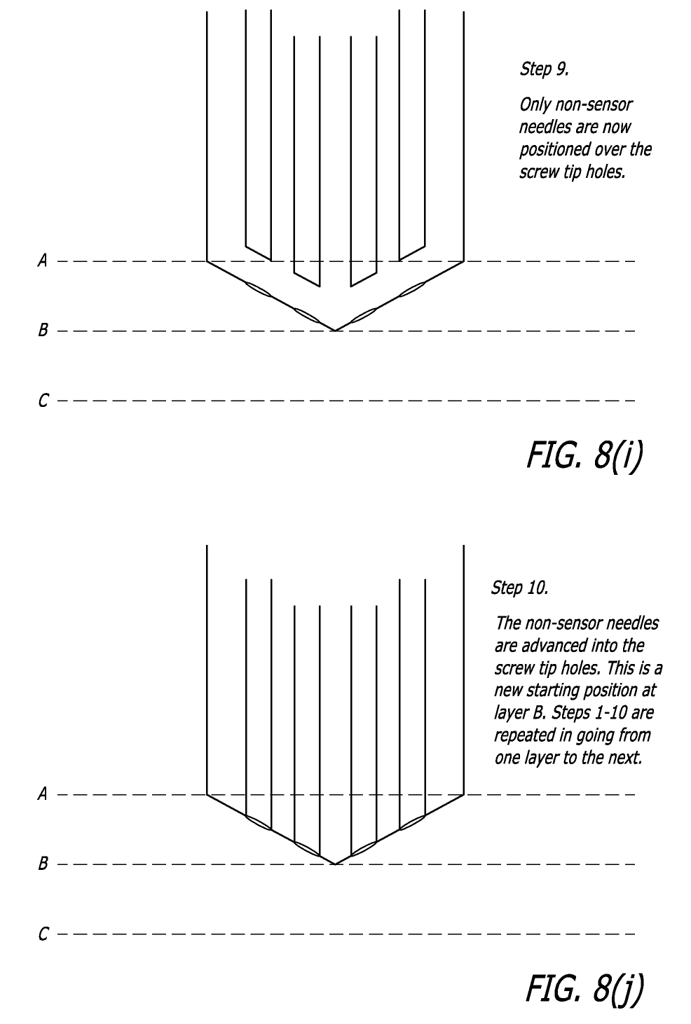

Artifactual noise can potentially be generated by ambient noise, by the use of a nearby electric drill, and by the crunching of bone at the device tip. Another potential problem is that compressive forces generated by bone on the device may create an axial strain that may affect the performance of the piezoelectric layer 15. To address these possible problems, the axial and rotation movement of the implantable instrument 10 may be separated from the acoustic measurement process, with the possible use of a shield to protect the transducer 12 during the advancement of the implantable instrument 10. This may be accomplished using the steps as illustrated in FIGS. 8(a)-8(j) discussed hereafter. An alternate embodiment to effect separation may use an alternating-movement shutter mechanism where the apertures are open when the acoustic measurement is made, and the apertures are closed when the acoustic measurement is not made.

As the transducer 12 is placed on the calcaneus, successive reflections from skin, cortex, and trabecular bone are shown in FIG. 3. The reflection from the cortex is much larger in amplitude than that of the marrow. For a backscatter-sensing transducer housed in a implantable instrument, small bony volumes in front of the device tip may be successively sampled and A-mode localized through sound reflections (backscatter), and characterized as to bone type (marrow versus cortex).

FIG. 3 illustrates how a time-gated pulse echo backscatter-sensing system operates in bone. Time gating allows the acquisition of the backscattered echoes from the region of interest. The backscattered echoes may be normalized to the echo from a flat reflector to minimize the effect of the experimental system on the data. Time gating allows the acquisition of the backscattered echoes from the region of interest. The backscattered echoes may be normalized to the echo from a flat reflector to minimize the effect of the experimental system on the data. Because of the small apertures used, optimal impedance matching may be required.

The selection of the transducer 12 may be based on performance characteristics. Several studies, largely from the osteoporosis literature, indicate that the 0.5-3.5 MHz frequency range is satisfactory for quantitative ultrasound through-transmission measurements that use two transducers placed on opposite sides of the calcaneus. In such a system, there may be plenty of working space available for the use of wide diameter transducers.

In spine surgery, the surgical work space is limited, and even more so in the hollow space inside implantable instruments such as bone screws. In adult humans, the typical dimensions of the inserted instruments can vary between 6-10 mm diameter and 35-60 mm in length. The medical device may have a hollow center diameter that is two-thirds of the outer device diameter.

Several different types of transducers 12 maybe used. For example, a pulsed unfocused transducer: As per Shung (Shung K K. Diagnostic Ultrasound: Imaging and Blood Flow Measurements. 2005, p. 62-67 and Shung K K et al., Ultrasonic Scattering by Biological Tissues. 1993).

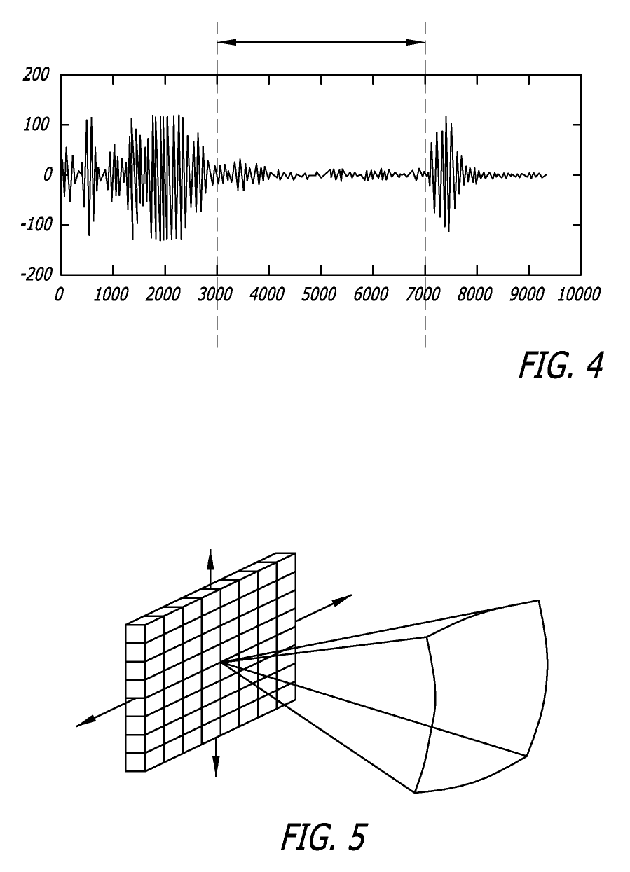

FIG. 4 shows the compact bone defined by the width of the bold-faced arrow, and bordered by regions of cancellous bone. Backscatter properties from biological tissues such as integrated backscatter and the slope of the backscatter may be used to characterize tissues (Shung). The backscattering properties are likely affected by the size and the acoustic properties of the scatterers relative to the surrounding medium. In addition, the statistical properties of the backscattered may also be used to characterize tissues.

As the scatterers in tissues are likely not completely randomly distributed, the backscattered echoes are likely better described by the K distribution and the Nakagami distribution (Weng et al., IEEE Trans Sonics Ultrason. 1983; SU-30, 156-183). The derived parameters from these statistical models are SNR, RMS and the m parameter of the Nakagami distribution where m=[E(A²)]²/{E[A²−E(A²)]². These parameters are calculated from the envelopes of the backscatter signals in a sample volume used for tissue characterization. The m parameter of 1 and 0.5, respectively, will result in Rayleigh distribution and half-Gaussian distribution. From the Wang-Tsai study it has been determined that bone tissue may be characterized on the basis of the parameters of the underlying statistical distribution, and can be useful in distinguishing between normal and osteoporotic bone.

Furthermore, as shown in FIG. 3, even with a 1 MHz unfocused transducer (Panametrics A303S), it may be possible to analyze backscatter data so as to determine a spatially distinct edge between bone tissue.

Therefore, transmittal by the transducer 12 of a signal indicative of the acoustic impedance of matter in the vicinity of the implantable instrument after it is implanted into the bone tissue and subsequently processed by the signal processing system based on the above discussed parameters, may be useful in distinguishing between bone tissue and thus useful in the guidance and positioning of implantable instruments into bone tissue.

Moreover, an unfocused transducer has the advantage of simplicity, but may waste relatively more of its sonic energy in regions not of immediate interest. By comparison, a focused transducer in the transmit mode may better direct the available energy into a small volume of bone tissue a few millimeters ahead of the screw tip, and result in a superior signal-to-noise ratio, with an improvement in the dB level of up to 10. However, a limitation of a single-element focused transducer may be that it has a fixed focal length with a small depth of focus in a single direction, which may be restrictive from the surgical standpoint. Because of diffraction limitations, critical structures outside the depth of focus, either too far or too close, may not be imaged or anticipated with a focused transducer, unless the aperture or the frequency is increased (Wang et al., IEEE Trans Biomed Eng 1997; 549-554). Either one of these two alternatives may have its drawbacks: the aperture is limited by the screw's hollow diameter, and increasing the frequency increases the attenuation. Similarly, beam spread may be reduced by increasing the frequency or using a larger element diameter or both.

Pulsed focused transducers have been used to demonstrate the variability of quantitative ultrasound parameters as a function of frequency, which can be improved by spatial averaging over the sample volume. (Hakulinen M A et al., Physics in Medicine and Biology 2005;50:1629-1642).

Short pulses have been frequently used in ultrasound to optimize axial resolution. In this particular application, if necessary, axial resolution may be sacrificed somewhat to increase the signal to noise ratio by transmitting more than one cycle of signals.

These four problems—focused vs. non-focused, rigid focal length, non-steerability, and forward vs. side-viewing—may be solved with the use of a multi-element microarray transducer.

As shown in FIG. 5, A 2-dimensional pyramidal array, offers a focusable beam (in the azimuthal and elevation directions) that provides a simultaneous mix of both forward and side imaging with improved contrast and better control of slice thickness. The point of focus along an axis of choice may be changed at will, thus allowing the study of different spatial volumes at distances far and near. This is one specific embodiment of a multi-element microarray system (MEMS), but other embodiments and configurations such as piezo electric micromachined ultrasonic transducers (pMUTS) are possible.

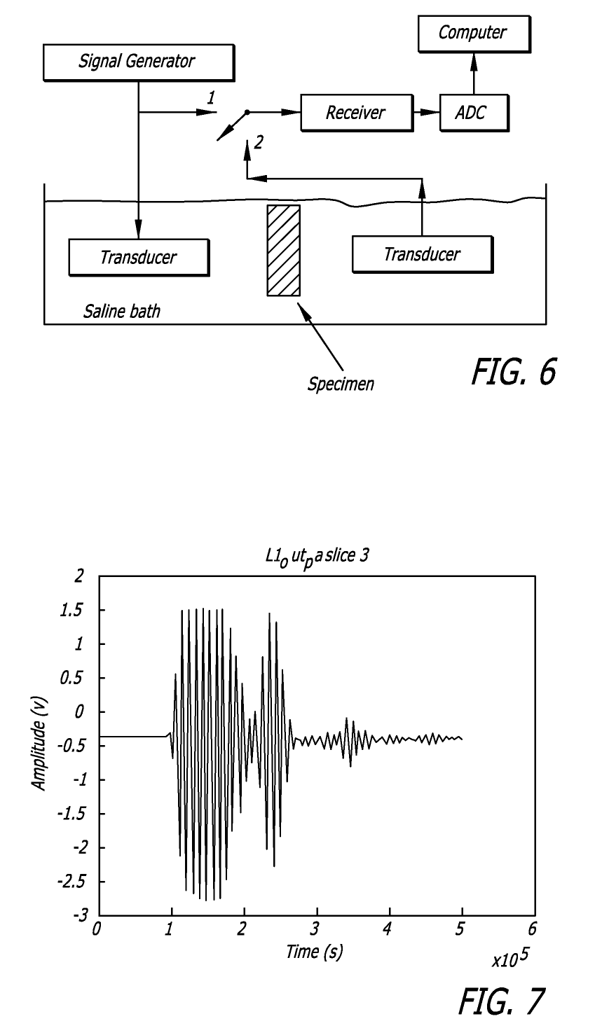

FIG. 6 illustrates an experimental set up that may be used to test the transducer. A water bath setup as shown in FIG. 6 to the left (switch position 1 for scattering measurements and 2 for velocity and attenuation measurements). A function generator or a pulser may be used to excite the transducer. A power amplifier may be used if the function generator alone does not yield sufficient signal to noise ratio. For the backscatter measurements, the returned echoes may be detected by the same transducer, whereas for the velocity of sound (VOS) and attenuation measurements the transmitted signals may be measured by a receiving transducer. The received signals may be amplified, digitized and acquired by a computer for calculating attenuation, velocity and backscattering parameters.

To distinguish between cortical and cancellous bone, the quantitative elastic properties of the tissue within the spatial volume of interest may be characterized (Nicholson et al., Phys Med Biol 1996; 41:2421-35 and Laugier P et al., Clin Rheum 1994; 13 (suppl 1), 22-32).

Broadband ultrasound attenuation (BUA), defined as the slope of the attenuation versus frequency curve, shows a strong increase of attenuation with frequency, which likely reflects increased scattering as the wavelength approaches the dimensions of the bone elements. The slope of the attenuation coefficient in cortical and cancellous bone, respectively, are approximately 5 and 10-40 dB cm⁻¹ MHz⁻¹ in comparison to soft tissue (the BUA for muscle is 0.5-1.5 dB cm⁻¹ MHz⁻¹). In cortical bone, there is a strong non-linear increase of attenuation, less in the axial than in the radial direction (Lakes R et al., J Biomed Eng 1986;8: 143-8 and Lees S, et al., Ultrasound Med Biol 1992; 18: 303-308).

In cancellous bone, BUA is a profoundly non-linear function of porosity, with BUA rising to a maximum at porosities of approximately 70% (Hodgskinson R, et al., Physics in Medicine & Biology 1996; 16:2411-2420).

Cortical bone is a relatively non-dispersive medium, such that the different velocities (group, phase, and signal) are numerically similar. (Hodgskinson R. et al., Bone 1997; 21: 281-285 and Roberjot V, et al., Osteoporosis 1997; 7:261).

Velocity of sound (VOS) and bone mineral density (BMD) are strongly correlated with the cortical elastic modulus (Lee S C. et al., Bone 1997; 21(1):119-125).

In cancellous bone, a strong linear relationship between velocity of sound (VOS ) and bone mineral density (BMD) exists over a wide range. As the screw advances parallel to the pedicle, the VOS measurement would likely be affected by the screw axis orientation relative to the major trabecular bone orientation. Propagation in cancellous bone is likely complicated by pulse broadening due to attenuation, which may lower the central frequency such that the signal velocity depends on the marker used to define transit time. A corrective approach may be to use phase spectral analysis of the broadband pulse to derive phase velocity and group velocity, as described by Strelitzki (Strelitzki R et al., Physiol Meas 1997; 18: 119-127 and Strelitzki R, et al., Phys Med Biol 1996;41: 743-53).

This attenuation effect may also be reduced by cross-correlation to produce a ‘mean pulse velocity’. In both cortical and cancellous bone, wave propagation is acoustically anisotropic, and the ultrasonic properties may vary with the direction in which the measurement is made. (Njeh C F et al., Osteoporosis Int 1997;7: 471-477 and Strelitzki R, et al., Eur J Ultrasound 1996; 4:205-13).

Using extremely small specimens (0.3 mm cross-section), Young's modulus has been shown to be significantly greater in cortical than in trabecular bone (21 versus 15 GPa, respectively) (Rho J Y, et al., J Biomech 1993; 26: 111-1119).

Using broadband ultrasound backscatter (BUB), backscatter coefficients in trabecular bone are frequency dependent, but with a flattened response at high frequencies (Wear K A. Frequency dependence of ultrasonic backscatter from human trabecular bone: theory and experiment. J Acoust Soc Amer 1999; 106(6):3659-3664 and Roux C, et al., J Bone Miner Res 2001;16:1353-1362).

When properly compensated for attenuation and geometrical factors, the backscatter coefficient depends only on the microstructure and acoustic properties of the scattering volume.

FIG. 7 illustrates A-mode monitoring of the peak associated with the marrow cortex edge may be used to prevent pedicle perforation. B-mode acoustic images of the bony tissue in the immediate vicinity of the screw tip may also be obtained.

An imageable B-mode difference between cortex and marrow can be obtained that may be used clinically. If the direction of the incident acoustic beam can be angled, it may be possible to create a contour surface map of the marrow-cortex interface, based upon the BUB peak distances obtained at different angles.

Method of Use of the Screw Insertion System

A method of use of the acoustic backscatter-sensing screw insertion system may consist of any or all of the following steps:
(1) Pre-Sizing the Screw Diameter
(2) Determining the Screw Direction
(3) Pre-Sizing the Screw Length
(4) Initialization of Outer Cortex and Marrow Acoustic BUB Values
(5) Detection of Distal Marrow-Cortex Edge
(6) Possible Separation of Screw Advancement from BUB sensing
(7) Detection of Complications
(8) Replacing the Acoustic Transducer with an Interlocking Core

Pre-Sizing the Screw Diameter

Intrapedicular Approach: In an example of insertion of a transducing implantable medical device 1, an intrapedicular approach may be used. In an intrapedicular approach, the usual order of the instruments used in the insertion of the implantable instrument 10, such as in screw insertion, may be: awl, pedicle probe, pedicle ‘feel’ probe, pedicle screw tap, and screw. If so desired, each of these instruments can be equipped with an internal transducer. The orientation points are the transverse process and the articular facet. The awl may create a start-up hole (about 0.5 cm deep) in the cortical bone at the midpoint of the base of the transverse process, and the probe may be angled approximately 45 degrees medial to the transverse process. The hole may then be extended by the pedicle probe for several centimeters (3-4 cm).

Next the hole may be explored and a ‘feel’ probe for possible perforations of the cortical rim. If no perforations are found, the pedicle screw tap may be used to drive the screw a short distance into the bone so that the screw threads are engaged, and to widen the hole. Finally, the screw may be manually advanced into the bone. After the preliminary hole is made, a side-viewing transducer may be introduced into the hole. At any given depth in the hole, a side-viewing transducer screw may be rotated through a screw rotation, and a 2-D B-mode image of the region around the screw may be obtained. In this manner the radial distance from the screw to the cortical rim may be determined. Successive 2-D B-mode scans (so called B-D scans) with a side-viewing transducer allow the creation of an equivalent 2-D contour of the outer surface, in a manner similar to BUA, but do not provide any forward imaging.

As such an improved system may simulate current surgical practice by creating a small-diameter drill hole and inserting a single side-imaging transducer probe. A transducer shaft, with a protractor attached, may be marked at 5 mm increments. At variable insertion depths (5 mm separation), a side-viewing transducer may be rotated through different angles, so as to generate the corresponding B-mode profile of the pedicle cortex rim. Multiple data sets per pedicle may be averaged to construct a 2-D B-mode contour of the pedicle rim.

In a microarray approach a multi-element microarray transducer may generate a simultaneous forward and side-viewing B-mode image of the marrow-cortex interface. The real-time B-mode 3-D contour map of the pedicle cortex would be an extremely useful aid to the surgeon in determining the position and depth of the implantable instrument.

Transpedicular Approach: In a transpedicular approach, the narrowest part of the pedicle (the pedicle neck) occurs anterior to the mid-point of the base of the transverse process (see FIG. 1a). Transverse placement of the transducer at the outer pedicle neck may make it possible to measure the pedicle's narrowest transverse diameter. By rotating a single transducer through an angular sector, it may become possible to image the medial cortex rim and thus to determine the width of the pedicle. Hence it may become possible to select the ideal width for the screw shaft. The narrower the pedicle neck, the narrower the chosen screw width. During screw advancement within a pedicle, the same external transducer left in place may be used to monitor for incipient plastic deformation of the pedicle and thus prevent its rupture. Multiple B-mode measurements about the pedicle neck allow a reconstruction of the pedicle neck in the form of a 2-D contour map. The drawback of this approach may be that, with the patient in the prone position, an extra surgical step is required to access the space anterior to the transverse process.

Moreover, another transpedicular approach may include a forward-looking transducer 12 which may be placed perpendicular to the long axis of the pedicle neck. With B-mode imaging, the transverse dimensions of the pedicle may be B-mode measured at different angles from various transducer positions on the pedicle circumference.

In a modified BUA imaging approach, an equivalent two-transducer BUA imaging system may be created from the use of an external transducer placed perpendicular to the outer pedicle neck, in conjunction with an intrapedicular side-viewing transducer. This combined two-transducer approach could be used to create an “X-ray”-like image of the pedicle.

Moreover, in an improved modified BUA imaging approach a similar method such as the improved intrapedicular approach may be used, except that a second external transducer may be placed perpendicular to the pedicle neck tip simultaneously to generate a BUA image.

Determining the Direction of the Screw

As discussed above, a 2-D contour map of the pedicle may be constructed from A-mode/B-mode data when a forward-looking transducer is placed on the pedicle neck (transpedicular) or when a side-viewing transducer is inserted into an initial hole drilled into the pedicle (intrapedicular). If the forward-looking transducer is placed at the screw entry point, it may be possible to angle the transducing screw so that the distance to the distal marrow-cortex edge in the vertebral body is maximized. Thus, the best direction for the screw advancement may be predetermined, by way of a 2-D contour map.

Sizing the Length of the Screw

An acceptable final position of the screw tip may be a few mm (<5 mm) from the marrow-cortex edge. To size the length of the screw, the distance from the outer pedicle screw entry point to the distal trabecular-cortex interface must be determined, minus a small safety margin. Adjunct signal processing with the Nakagami distribution may be required to highlight the marrow-cortex edge.

A forward-looking transducer 12 may be placed medial to the base of the mid-transverse process in the adjacent groove, and may be aligned with the known axis of the pedicle. This may allow estimation of the screw length prior to the screw's advancement.

Initialization of Outer Cortex and Marrow Acoustic BUB Values

With the sensing implantable instrument 10 atop the vertebral bone, the broadband ultrasound backscattering (BUB) and elastic parameters from the proximal cortical bone may be measured, so as to establish expected quantitative cortex values. Integrated backscatter, slope of the backscatter as a function of frequency, elastic modulus (EM), and statistical parameters, e.g., the Nakagami index may be calculated. The implantable instrument 10 may then be advanced through the proximal cortex-marrow interface, that is, from cortical bone (higher backscattering) and then into the cancellous bone (lower backscattering). The BUB and EM quantitative values for cancellous bone may be determined. The reference BUB and EM values for cortical and cancellous bone may thus be established during the initialization phase.

Once past the proximal cortex, the device may be advanced through the pedicle without penetrating distal cortex.

Marrow-Cortex Edge Detection

With time-gated pulse-echo ultrasound, selected spatial volumes ahead of the implant device tip may be analyzed for backscatter. As the instrument tip advances through cancellous bone, a long plateau is likely encountered where the BUBs and EMs are lower-valued. As distal cortex is approached, the BUB and EM measurements may get stronger, comparable to those seen during the initialization phase. The A-mode amplitude likely increases, by serving as an indication of close proximity to cortex, to the point of saturation (see FIG. 7 to left). Both A-mode and BUB readings can be thresholded to serve as quantitative alarms.

Possible Separation of Screw Advancement from BUB Sensing

Artifactual noise can be generated by ambient noise, or by the crunching of bone at the screw tip. A potential problem may be that compressive forces generated by bone on the device create an axial strain that affects the electrical resistivity of the piezoelectric layer.

Possible design solutions include recessing the transducer within the screw, providing a protective sonic shield, or completely separating the axial and rotation movement of the implantable instrument 10, from the acoustic measurement process.

The latter may require the repeated use of four separate steps-activation of a transducer shield during axial advancement and rotation of the implantable instrument.

FIGS. 8(a)-8(j) illustrate a method that may separate the axial and rotation movement of the implantable instrument 10, from the acoustic measurement process.

FIG. 8(a) shows the implantable instrument 10 in a starting position with the distal tip of the implantable instrument 10 at layer A of the bone tissue. As illustrated in FIG. 8(a), the implantable instrument may include a plurality of apertures 51 on the distal tip of the implantable instrument 10. The internal chamber of the implantable instrument 10 may include solid core components such as non-sensing beveled needles 53 that may fill and align with the plurality of apertures 51.

FIG. 8(b) shows the how the implantable instrument 10, such as a screw may be rotated and advanced through bone tissue so that the tip of the screw advances to a second layer B, of the bone tissue.

FIG. 8(c) shows a third step in the method of advancement of the implantable instrument 10 so that the axial and rotation movement of the implantable instrument may be separated, and in which the non-sensing beveled needles 53 are retracted from the internal chamber of the implantable instrument 10.

FIG. 8(d) shows a fourth step in which the non-sensing beveled needles 53 are retracted from the internal chamber of the implantable instrument 10 and replaced with transducer components 12 which are configured to generate an signal indicative of the acoustic impedance of matter in the vicinity of the implantable instrument 10.

FIG. 8(e) shows complete replacement of the non-sensing beveled needles 53 with transducer components 12.

FIG. 8(f) shows the advancement of the transducer components 12 through the plurality of apertures 51 so that the transducer components 12 are advanced to layer C.

FIG. 8(g) shows the retraction of the transducer components 12 from the plurality of apertures 51.

FIG. 8(h) shows a seventh step in which the transducer components 12 are retracted from the internal chamber of the implantable instrument 10 and replaced with transducer non-sensing beveled needles 53 as the implantable instrument is rotated.

FIG. 8(i) shows the position of the distal tip of implantable instrument 10 at layer B and with non-sensing beveled needles 53 withdrawn in the internal chamber of the implantable instrument 10.

FIG. 8(j) shows the position of the distal tip of implantable instrument 10 at layer B and with non-sensing beveled needles 53 extended in the internal chamber of the implantable instrument 10 so that the plurality of apertures 51 are filled by the non-sensing beveled needles 53.

These steps may be repeated until the implantable instrument 10 reaches the desired depth and position in the bone tissue.

Once the implantable instrument 10 has been positioned at the desired location and depth, the transducer 12 may be removed from the internal chamber of the implantable instrument 10 and replaced with a solid mass. The transducer 12 may then be re-used in another implantable instrument.

Detection of Complications

The signal processing system 40 may include a computer-based analyzer, the transducer signal may be inputted, and the computer may compare the measured elastic moduli (EM) at each position versus the allowed EM thresholds established during the initialization phase. A continuous display of the BUB signal peak vs. screw tip insertion distance plot may be made.

A visual alarm may be displayed on a screen indicating that the distal BUB peak is too close to the tip of the implantable instrument, or the distal BUB peak numerical value approaches that of the initialized BUB peak value, thus informing the operator to stop advancing the instrument.

Alternatively or in addition to the visual alarm, an audible alarm may be sounded when the BUB peak is too close to the tip of the implantable instrument, thus informing the operator to stop advancing the instrument.

A break or discontinuity in the image of the distal marrow-cortex interface may indicate a pedicle cortex perforation, thus informing the operator to withdraw the screw.

An image of hypoechoic fascicles within an enclosed structure may indicate that the screw tip is in the vicinity of spinal nerve roots in the epidural space, or other key paraspinal neural structures, including the spinal cord, thus informing the operator to withdraw the screw.

An image of a pulsating or easily collapsible vessel may indicate the proximity of a spinal cord vessel, either an artery (pulsating) or a vein (easily collapsible), thus informing the operator to withdraw the screw.

An image of a hypoechoic layer underneath a bright layer may indicate the nearby presence of the subarachnoid space containing cerebrospinal fluid, and of the underlying spinal cord, thus informing the operator to withdraw the screw.

Replacing the Acoustic Transducer with an Interlocking Core

Upon advancement of the screw into its final position, the transducer may be removed from the hollow center of the screw, and may be replaced with a solid interlocking same-shaped piece.

In brief, a method is presented by which to improve the efficacy and safety of orthopedic implant insertion procedures, and to markedly reduce complications, particularly during spine surgery. A specific and immediate concern arises in spine transpedicular fixation surgery, where a screw can either rupture the pedicle wall, or be advanced too far into the vertebral body, and can cause damage to the nearby spinal cord and spinal nerve roots. This concern continues, despite the use of a variety of imaging approaches involving radiation that basically image the bone from afar in order to ascertain screw tip location (radiographs, fluoroscopy, CAT scans), or that infer screw tip proximity to a damageable neural structure based upon local electrical nerve stimulation (electromyography). We propose that surgical implant devices (screws, awls, screw taps, ‘feeler’ probes, rods, pins) can be equipped with an acoustic pulse-echo backscatter-sensing capability, so that “the surgeon can see what the device tip sees.” Such operator-based instrument vision improves guidance, achieves safe and exact device positioning, and reduces the need for ionizing radiation.

The proposed method, in one embodiment, involves an acoustic time-gated, pulse echo, backscatter-sensing transducer system, coaxially housed within a screw, which can perform A- and B-mode imaging and elastic characterization of bone tissue in front of and around the transducer tip. Such an instrument insertion system with acoustic backscatter capabilities can be used in transpedicular spine fixation surgery by way of pre-operatively sizing pedicle screws, estimating distances to bone tissue interfaces, guiding the inserted screw, and detecting complications,

The fixation or removal of the transducer may be accomplished with a mechanical fastening mechanism.

The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

The terms “transmit” and “receive” encompass both direct and indirect transmittal and reception. For example, the terms “transmit” and “receive” encompass the presence of intervening signal transmittal between the transmitting and receiving components.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

We claim:

1. A medical device comprising:
an implantable instrument configured to be implanted in bone tissue, the implantable instrument having an internal chamber configured to house at least one transducer;
a transducer located within the internal chamber of the implantable instrument, the transducer configured to transmit a signal and to receive one or more reflections of that signal; and
a signal processing system configured to process the signals transmitted and received by the transducer so as to provide information indicative of the acoustic impedance of matter in the vicinity of the implantable instrument after it is implanted into the bone tissue based on the signals transmitted and received by the transducer.

2. The medical device of claim 1, wherein the transducer includes piezoelectric material.

3. The medical device of claim 1, wherein the transducer is an acoustic transducer.

4. The medical device of claim 1, wherein the medical device is configured to compensate for axial strain generated by bone tissue during advancement of the implantable instrument.

5. The medical device of claim 4 configured to compensate for axial strain generated by bone tissue during advancement of the implantable instrument, wherein the implantable instrument includes a plurality of apertures at the distal tip of the implantable instrument.

6. The medical device of 5, wherein the plurality of apertures are arranged to receive a plurality of removable needles upon advancement of the medical instrument through bone tissue; a plurality of needles configured to be aligned with the surface of the distal tip of the medical instrument.

7. The medical device of claim 5 wherein the transducer is configured to be advanced through the plurality of apertures.

8. The medical device of claim 1, wherein the transducer includes piezoelectric material, a backing layer and one or more matching layers configured to increase signal sensitivity and a lens.

9. The medical device of claim 1, wherein the implantable instrument is made of a biocompatible material.

10. The medical device of claim 9, wherein the biocompatible material is titanium.

11. The medical device of claim 1, wherein the signal processing system includes a communication device configured to communicate information indicative of the acoustic impedance of matter in the vicinity of the implantable instrument after it is advanced into the bone tissue, and a quantitative alarm configured to indicate the depth of the tip of the implantable instrument.

12. The medical device of claim 1, wherein the signal processing system includes a communication device configured to communicate information indicative of the acoustic impedance of matter in the vicinity of the implantable instrument after it is advanced into the bone tissue, and an alarm configured to indicate when the implantable instrument is approaching an undesired depth.

13. The medical device of claim 1, wherein the implantable instrument comprises smooth external surface having a pointed tip configured to form holes in bone tissue.

14. The medical device of claim 1, wherein the implantable instrument comprises a threaded external wall.

15. The medical device of claim 1, wherein the transducer includes an array of transducers.

16. The medical device of claim 15 wherein the array of transducers is configured to generate a focusable beam to generate simultaneous forward and side imaging.

17. The medical device of claim 1, wherein the transducer is configured to focus pulsed energy ahead of the tip of the implantable device to generate forward image of the surrounding bone tissue.

18. The medical device of claim 1, wherein the transducer is configured to focus energy to the side of the tip of the implantable device to generate an image of the surrounding bone tissue to the side of the implantable device.

19. The medical device of claim 1, wherein the transducer is configured to transmit a signal between 0.5-3.5 MHz.

20. The medical device of claim 1, wherein the transducer is configured to be removed from the internal chamber and replaced with a solid cylindrical mass.

21. A medical device comprising an implantable instrument configured to be implanted in bone tissue, the implantable instrument having an internal chamber configured to house a transducer.

22. The medical device of claim 21, wherein the internal chamber of the implantable instrument includes a proximal and a distal end, wherein the proximal end is configured to channel an electrical lead from the transducer, and the distal end is configured to house the transducer.

23. The medical device of claim 22, wherein the proximal end of the electrical lead is configured to interface with an advancement device;
an advancement device configured to advance the medical instrument through bone tissue, wherein the advancement device is configured to transmit the electrical signal from the transducer.

24 The medical device of claim 21, wherein the implantable instrument includes an external thread surface configured to engage bone.

25 The medical device of claim 21, wherein the implantable instrument is made of a biocompatible material.

26 The medical device of claim 25, wherein the biocompatible material comprises titanium.

27. The medical device of claim 21, further comprising a solid cylindrical mass configured to fill the internal chamber when the transducer is not present.

28. The medical device of claim 21, wherein the implantable instrument is a bone screw.

29. The medical device of claim 21, wherein the implantable instrument is a bone awl.

30. The medical device of claim 21, wherein the implantable instrument is a pedicle probe.

31. The medical device of claim 21, wherein the implantable instrument includes a lens configured to be housed at the distal end of the internal chamber.

32. The medical device of claim 21, wherein the implantable instrument is configured to allow the transducer to be removed and replaced with a solid cylindrical mass.

33. A medical device comprising a transducer configured to be inserted within an internal chamber of an implantable instrument, the transducer configured to transmit a signal and to receive one or more reflections of that signal based on the signals transmitted by the transducer.

34. The medical device of claim 33, wherein the transducer includes piezoelectric material, a backing layer, one or more matching layers and a lens.

35. The medical device of claim 33wherein the transducer is an acoustic transducer.

36 The medical device of claim 35 wherein the transducer is all ultrasonic transducer.

37. The medical device of claim 33, wherein the transducer is configured to transmit a signal between 0.5-3.5 MHz.

38. The medical device of claim 33, wherein the transducer comprises a multi-element microarray transducer.

39. The medical device of claim 33, wherein the transducer comprises a lens.

40. The medical device of claim 33, wherein the lens comprises a material having an acoustic impedance similar to that of the bone.

41. A medical device comprising a signal processing system configured to process signals transmitted and received by a transducer that is inserted within an internal chamber of an implantable instrument, so as to provide information indicative of the acoustic impedance of matter in the vicinity of the implantable instrument after it is implanted into the bone tissue based on the signals transmitted and received by the transducer.

42. The medical device of claim 41, wherein the signal processing system includes a display configured to indicate the acoustic impedance of the matter in the vicinity of the implantable instrument after it is implanted into the bone tissue and advanced into the bone tissue, and a quantitative alarm configured to indicate the depth of the tip of the implantable instrument.

43. The medical device of claim 41, wherein the signal processing system includes a communication device configured to communicate information indicative of the acoustic impedance of matter in the vicinity of the implantable instrument after it is advanced into the bone tissue, and an alarm configured to indicate when the implantable instrument is approaching an undesired depth.

44. The medical device of claim 43, wherein the alarm is a audible alarm.

45. The medical device of claim 43, wherein the alarm is a visual alarm.

46. The device of claim 41, wherein the signal processing system is configured to generate reference value for the acoustic impedance of matter in the vicinity of the implantable instrument as it is advanced through bone tissue.

47. The device of claim 46, wherein the reference values are used to calculate the position of the implantable instrument.

48. The medical device of claim 41, further comprising a visual display, wherein the visual display, displays an image of the bone tissue in the vicinity of the implantable instrument.

49. The medical device of claim 48, wherein the image is a contour surface map of the bone tissue in the vicinity of the implantable instrument.

50. A method of implanting an implantable instrument into bone tissue, comprising the steps of:
inserting the implantable instrument into bone tissue having a transducer housed therein; and
adjusting the position of the implantable instrument based on an electrical signal from the transducer.

51. The method of claim 50 further comprising:
removing the transducer from the internal chamber of the implantable instrument, and
inserting a solid mass into the internal chamber.

52. The method of claim 50 further comprising transmitting an alarm when the implantable instrument is at a desired depth.

53. A method of using a medical device comprising:
an implantable instrument configured to be implanted in bone tissue, the implantable instrument having an internal chamber configured to house at least one transducer;
a transducer located within the internal chamber of the implantable instrument, the transducer configured to transmit a signal and to receive one or more reflections of that signal; and a signal processing system configured to process the signals transmitted and received by the transducer so as to provide information indicative of the acoustic impedance of matter in the vicinity of the implantable instrument after it is implanted into the bone tissue based on the signals transmitted and received by the transducer,
wherein, said method comprises the steps of:
a. pre-sizing the diameter of the implantable instrument,
b. determining the direction of the implantable instrument,
c. pre-sizing the length of the implantable instrument,
d. initializing the transducer to transmit acoustic broadband ultrasound backscatter values obtained for the outer cortex and marrow,
e. configuring the signal processing system to detect the edge of the distal marrow-cortex,
f. configuring the medical device to separate advancement of the implantable instrument from values transmitted by the transducer,
g. monitoring the signal processing system for complications,
h. removing the transducer from the internal chamber of the implantable instrument and inserting an interlocking core in the internal chamber.

54. The method of claim 53, wherein the complications of step g comprise: cortex perforation, and needle injury to key paraspinal structures.
